(19) United States
(12) Patent Application Publication
(10) Pub. No.: US 2016/0374929 A1
(43) Pub. Date: Dec. 29, 2016

(54) MAKE-UP COSMETIC COMPOSITION CONTAINING MQ SILICONE RESIN AND PROPYL SILSESQUIOXANE RESIN

(71) Applicant: AMOREPACIFIC CORPORATION, Seoul (KR)

(72) Inventors: Hye Jeon CHO, Yongin-si (KR); Kyung Nam KIM, Yongin-si (KR); Kyung Ho CHOI, Yongin-si (KR); Yeong Jin CHOI, Yongin-si (KR)

(21) Appl. No.: 15/258,357

(22) Filed: Sep. 7, 2016

Related U.S. Application Data
(63) Continuation-in-part of application No. 14/398,013, filed on Oct. 30, 2014, now Pat. No. 9,463,152.

(30) Foreign Application Priority Data
Apr. 30, 2012 (KR) 10-2012-0045406
Apr. 19, 2013 (KR) 10-2013-0043591

(51) Int. Cl. A61K 8/891; A61K 8/19; A61K 8/81; A61K 8/29; A61Q 1/00; A61Q 19/00

(52) U.S. Cl. CPC A61K 8/891; A61Q 1/00; A61Q 19/00; A61Q 19/007; A61K 8/8152; A61K 8/29; A61K 8/19; A61K 2800/594

(57) ABSTRACT

The present invention relates to a make-up cosmetic composition containing MQ silicone resin and propyl silsesquioxane resin, and, more specifically, relates to a make-up cosmetic composition which contains MQ silicone resin and propyl silsesquioxane resin, gives an outstanding moisturising impression and is free from any pulling phenomenon.

This application is a Continuation-in-part application of U.S. patent application Ser. No. 14/398,013, which is the U.S.A. national phase entry of International Application No. PCT/KR2013/003412, filed 22 Apr. 2013, which designated the U.S. and claims priority to KR Application No. 10-2012-0045406, filed 30 Apr. 2012, and KR Application No. 10-2013-0043591, filed 19 Apr. 2013 and is also now allowed on 6 Jul. 2016, the entire contents of each of which are hereby incorporated by reference.

### TECHNICAL FIELD

The present invention relates to a makeup cosmetic composition containing, as active ingredients, MQ silicone resin and propyl silsesquioxane resin, which make the skin moist without drying or pulling of the skin.

### BACKGROUND ART

With the passage of time after application of a makeup cosmetic composition such as a foundation to the skin, the removal of makeup by the sweat and sebum secreted from the skin is observed. In a method for maintaining a makeup layer for a long time, MQ silicone resin can be used to improve the maintenance of a pigment or other active materials on the skin, but cosmetic compositions comprising MQ silicon resin have the problem of causing drying and pulling of the skin. For this reason, there is a need for a siloxane resin that exhibits the maintenance similar to that of MQ silicone resin used in current cosmetic formulations and that exhibits an excellent moisturizing sensation and does not cause pulling of the skin.

### DISCLOSURE

### Technical Problem

As used herein, “pulling of the skin” refers to a phenomenon that can easily occur when moisturization of the skin or the supply of moisture to the skin is insufficient. The present inventors have found that MQ silicone resin and propyl silsesquioxane resin have excellent effects of preventing pulling of the skin and moisturizing the skin, thereby completing the present invention.

Therefore, it is an object of the present invention to provide a makeup cosmetic composition containing MQ silicone resin and propyl silsesquioxane resin, which exhibit an excellent moisturizing sensation and do not cause pulling of the skin.

### Technical Solution

The present invention is directed to a makeup cosmetic composition containing, as active ingredients, MQ silicone resin and propyl silsesquioxane resin, which make the skin moist without drying or pulling of the skin.

### Advantageous Effects

A makeup cosmetic composition according to the present invention can form a long-lasting makeup layer and maintain the skin in a moist state without drying or pulling of the skin.

### DESCRIPTION OF DRAWINGS

FIG. 1 is a set of photographs showing a state in which compositions of Comparative Example 4 and Example 8 were applied to synthetic leather (FIG. 1a) and a state in which the leather was pulled after 15 minutes of drying following application of the compositions (FIG. 1b).

FIG. 2 is a set of photographs showing a state in which compositions of Comparative Example 4 and Example 8 to 14 were applied to synthetic leather.

### BEST MODE

The present invention relates to a makeup cosmetic composition containing, as active ingredients, MQ silicone resin and propyl silsesquioxane resin, which make the skin moist without drying or pulling of the skin.

The present invention will be described in further detail.

In an embodiment of the present invention, the MQ silicone resin is selected from among an M unit represented by (R₃¹SiO_{1/2})ₐ and a Q unit represented by (SiO_{4/2})_{b}, wherein the R¹ groups may independently be an alkyl group having 1 to 8 carbon atoms or an aryl group. Examples of the alkyl group include methyl, ethyl, propyl, butyl, pentyl and hexyl, and examples of the aryl group include phenyl, naphthyl, benzyl, tolyl, xylyl and xenyl. a and b are integers greater than 0, and the ratio of a/b is 0.5-1.5. The MQ silicone resin may further contain at least one of a D unit and a T unit, provided that at least 80 mole % or at least 90 mole % of the MQ silicone resin consists of the M and Q units. The MQ silicone resin may be, for example, trimethylsiloxysilicate, but is not limited thereto.

In an embodiment of the present invention, the propyl silsesquioxane resin comprises a T unit, represented by R²SiO_{3/2}, in an amount of at least 80 mole % based on the total moles of the propyl silsesquioxane resin. Herein, the R² groups are independently an alkyl group having 1 to 8 carbon atoms or an aryl group, and at least 40 mole % of R² is propyl. The propyl silsesquioxane resin may further contain one or more from the group consisting of M, D and Q units, provided that at least 80 mole % of the propyl silsesquioxane resin consists of the T units. The propyl silsesquioxane resin may be, for example, polypropylsilsesquioxane.

The M unit, D unit, T unit and Q unit are silicone polymer units that may be represented by the following formula 1:

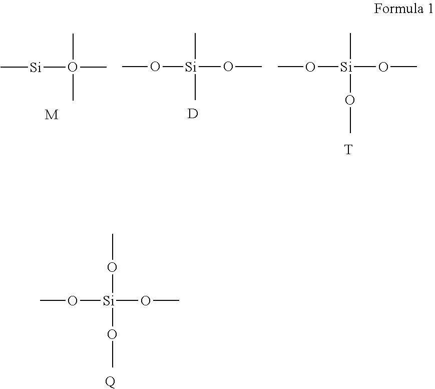

In an embodiment of the present invention, the MQ silicone resin may be contained in an amount of 0.01-30 wt %, preferably 0.01-10 wt %, and most preferably 0.01-7.5 wt %, based on the total weight of the composition. If the content of the MQ silicone resin in the composition is less than 0.01 wt %, it will not significantly contribute to skin moisturization, and if the content of the MQ silicone resin is more than 30 wt %, it will cause severe pulling of the skin.

In an embodiment of the present invention, the propyl silsesquioxane resin may be contained in an amount of 0.01-30 wt %, preferably 0.01-10 wt %, and most preferably 0.01-7.5 wt %, based on the total weight of the composition. If the content of the propyl silsesquioxane resin in the composition is less than 0.01 wt %, it will not significantly contribute to skin moisturization, and if the content of the propyl silsesquioxane resin is more than 30 wt %, the touch feeling of the composition can become heavy.

In an embodiment of the present invention, the composition may contain the MQ silicone resin and the propyl silsesquioxane resin at a weight ratio of 1:150 to 150:1, preferably 1:80 to 80:1, more preferably 2:75 to 75:2, even more preferably 1˜3:1˜3, even more preferably 1:1, 1:3, 1:2, 2:3, 3:2, 2:1 and 3:1, and most preferably 1:1, 1:3, 1:2 and 2:3. If the weight ratio is out of the above range, the touch feeling of the composition can be reduced.

A composition according to an embodiment of the present invention may contain a cosmetically or dermatologically acceptable medium or base. It may preferably be prepared into any formulation suitable for topical application and may be of a type selected from the group consisting of a water-in-oil type, an oil-in-water type and an oil dispersion type. Also, it can be provided in the form of suspension, microemulsion, microcapsule, microgranule or ionic (liposome) or non-ionic vesicular dispersion. In addition, it can be provided in the form of cream, skin toner, lotion, powder, ointment, spray or conceal stick. In addition, it can be used in the form of foam or as an aerosol composition further containing a compressed propellant. These compositions can be prepared according to any conventional method known in the art.

A makeup cosmetic composition according to an embodiment of the present invention may contain additives that are generally used in the cosmetic or skin science field, as long as these additives do not impair the purpose of the present invention. Examples of these additives include fatty substances, organic solvents, solubilizing agents, thickeners, gelling agents, softeners, antioxidants, suspending agents, stabilizers, foaming agents, aromatic substances, surfactants, water, ionic or non-ionic emulsifying agents, fillers, sequestering agents, chelating agents, preservatives, vitamins, blockers, moisturizing agents, essential oil, dyes, pigments, hydrophilic or hydrophobic activators, lipid vesicles, or other components, which are generally used in cosmetics. The additives are contained in amounts that are generally used in the cosmetic or skin science field. Further, the composition of the present invention may contain a skin absorption-promoting material in order to increase the effects of improving skin conditions

### Mode for Invention

Hereinafter, the present invention will be described in further detail with reference to examples. It will be obvious to those skilled in the art that these examples are for illustrative purposes only and are not intended to limit the scope.

### COMPARATIVE EXAMPLES 1 TO 3 AND EXAMPLE 1

According to the components and contents shown in Table 1, Table 2 and Table 3 below, compositions of Comparative Examples 1 to 3 and Example 1 to 7 were prepared.

TABLE 1

| | | Comp. | Comp. | Comp. | |
|---|---|---|---|---|---|
| | | Exam- | Exam- | Exam- | Exam- |
| Components (wt %) | | ple 1 | ple 2 | ple 3 | ple 1 |
| Oil | Ethylhexyl | 5.00 | 5.00 | 5.00 | 5.00 |
| phase | methoxycinnamate | | | | |
| | Cyclopentasiloxane | 25.00 | 15.00 | 15.00 | 15.00 |
| | Methyl trimethicone | 5.00 | 5.00 | 5.00 | 5.00 |
| | Phenyl trimethicone | 4.00 | 4.00 | 4.00 | 4.00 |
| | Trimethylsiloxysilicate | — | 10.00 | — | 5.00 |
| | Polypropyl | — | — | 10.00 | 5.00 |
| | silsesquinoxane | | | | |
| | PEG-10 dimethicone | 3.50 | 3.50 | 3.50 | 3.50 |
| | Cetyl PEG/PPG-10/1 | 1.00 | 1.00 | 1.00 | 1.00 |
| | dimethicone | | | | |
| | Disteardimonium | 1.50 | 1.50 | 1.50 | 1.50 |
| | hectorite | | | | |
| Pow- | Titanium oxide surface | 8.00 | 8.00 | 8.00 | 8.00 |
| dery | treated with alkylsilane | | | | |
| mate- | Iron oxide yellow | 0.60 | 0.60 | 0.60 | 0.60 |
| rials | surface treated with | | | | |
| | alkylsilane | | | | |
| | Iron oxide red surface | 0.20 | 0.20 | 0.20 | 0.20 |
| | treated with alkylsilane | | | | |
| | Iron oxide black | 0.15 | 0.15 | 0.15 | 0.15 |
| | surface treated with | | | | |
| | alkylsilane | | | | |
| | Polymethylmethacrylate | 1.05 | 1.05 | 1.05 | 1.05 |
| Water | Purified water | 38.70 | 38.70 | 38.70 | 38.70 |
| phase | Butylene glycol | 5.00 | 5.00 | 5.00 | 5.00 |
| | Sodium chloride | 1.00 | 1.00 | 1.00 | 1.00 |
| | Phenoxyethanol | 0.30 | 0.30 | 0.30 | 0.30 |
| Weight | Trimethylsiloxysili- | — | — | — | 1:1 |
| ratio | cate:Polypropyl | | | | |
| | silsesquinoxane | | | | |

TABLE 2

| | | Exam- | Exam- | Exam- | Exam- |
|---|---|---|---|---|---|
| Components (wt %) | | ple 2 | ple 3 | ple 4 | ple 5 |
| Oil | Ethylhexyl | 5.00 | 5.00 | 5.00 | 5.00 |
| phase | methoxycinnamate | | | | |
| | Cyclopentasiloxane | 15.00 | 15.00 | 15.00 | 15.00 |
| | Methyl trimethicone | 5.00 | 5.00 | 5.00 | 5.00 |
| | Phenyl trimethicone | 4.00 | 4.00 | 4.00 | 4.00 |
| | Trimethylsiloxysilicate | 2.50 | 3.33 | 4.00 | 6.00 |
| | Polypropyl | 7.50 | 6.67 | 6.00 | 4.00 |
| | silsesquinoxane | | | | |
| | PEG-10 dimethicone | 3.50 | 3.50 | 3.50 | 3.50 |
| | Cetyl PEG/PPG-10/1 | 1.00 | 1.00 | 1.00 | 1.00 |
| | dimethicone | | | | |
| | Disteardimonium | 1.50 | 1.50 | 1.50 | 1.50 |
| | hectorite | | | | |
| Pow- | Titanium oxide surface | 8.00 | 8.00 | 8.00 | 8.00 |
| dery | treated with alkylsilane | | | | |
| mate- | Iron oxide yellow | 0.60 | 0.60 | 0.60 | 0.60 |
| rials | surface treated with | | | | |
| | alkylsilane | | | | |
| | Iron oxide red surface | 0.20 | 0.20 | 0.20 | 0.20 |
| | treated with alkylsilane | | | | |
| | Iron oxide black | 0.15 | 0.15 | 0.15 | 0.15 |
| | surface treated with | | | | |
| | alkylsilane | | | | |
| | Polymethylmethacrylate | 1.05 | 1.05 | 1.05 | 1.05 |
| Water | Purified water | 38.70 | 38.70 | 38.70 | 38.70 |
| phase | Butylene glycol | 5.00 | 5.00 | 5.00 | 5.00 |
| | Sodium chloride | 1.00 | 1.00 | 1.00 | 1.00 |
| | Phenoxyethanol | 0.30 | 0.30 | 0.30 | 0.30 |
| Weight | Trimethylsiloxysili- | 1:3 | 1:2 | 2:3 | 3:2 |
| ratio | cate:Polypropyl | | | | |
| | silsesquinoxane | | | | |

TABLE 3

| | | Example | Example |
|---|---|---|---|
| Components (wt %) | | 6 | 7 |
| Oil phase | Ethylhexyl | 5.00 | 5.00 |
| | methoxycinnamate | | |
| | Cyclopentasiloxane | 15.00 | 15.00 |
| | Methyl trimethicone | 5.00 | 5.00 |
| | Phenyl trimethicone | 4.00 | 4.00 |
| | Trimethylsiloxysilicate | 6.67 | 7.50 |
| | Polypropyl silsesquinoxane | 3.33 | 2.50 |
| | PEG-10 dimethicone | 3.50 | 3.50 |
| | Cetyl PEG/PPG-10/1 | 1.00 | 1.00 |
| | dimethicone | | |
| | Disteardimonium hectorite | 1.50 | 1.50 |
| Powdery | Titanium oxide surface | 8.00 | 8.00 |
| materials | treated with alkylsilane | | |
| | Iron oxide yellow surface | 0.60 | 0.60 |
| | treated with alkylsilane | | |
| | Iron oxide red surface | 0.20 | 0.20 |
| | treated with alkylsilane | | |
| | Iron oxide black surface | 0.15 | 0.15 |
| | treated with alkylsilane | | |
| | Polymethylmethacrylate | 1.05 | 1.05 |
| Water | Purified water | 38.70 | 38.70 |
| phase | Butylene glycol | 5.00 | 5.00 |
| | Sodium chloride | 1.00 | 1.00 |
| | Phenoxyethanol | 0.30 | 0.30 |
| Weight | Trimethylsiloxysili- | 2:1 | 3:1 |
| ratio | cate:Polypropyl | | |
| | silsesquinoxane | | |

Preparation Method

1) The above-described oil-phase components and powdery materials were mixed with one another to form a dispersion.

2) The water-phase components were mixed with one another.

3) The mixture of 2) was added slowly to the mixture of 1), and then stirred to form an emulsion.

4) The mixture was completely degassed, thereby preparing a water-in-oil type makeup cosmetic composition.

### TEST EXAMPLE 1 TO 7

### Sensory Evaluation of Makeup Cosmetic Compositions

Each of the compositions of Comparative Examples 1 to 3 and Example 1 to 7 was applied to the face of each of 20 women aged 25-39 years old, and after 12 hours, two items (moist sensation and no skin pulling) were evaluated by questionnaire. Each of the items was evaluated on a 5-point scale, and a better effect was given a higher score. The averages of the scores are shown in Table 4, Table 5 and Table 6 below.

TABLE 4

| | Compar- | Compar- | Compar- | |
|---|---|---|---|---|
| | ative | ative | ative | |
| Evaluation item | Example 1 | Example 2 | Example 3 | Example 1 |
| Moist sensation | 1.4 | 1.7 | 1.9 | 4.3 |
| without drying | | | | |
| No skin pulling | 1.3 | 1.5 | 1.8 | 4.2 |
| Weight ratio | — | — | — | 1:1 |
| (Trimethylsiloxysili- | | | | |
| cate:Polypropyl | | | | |
| silsesquinoxane) | | | | |

TABLE 5

| Evaluation item | Example 2 | Example 3 | Example 4 |
|---|---|---|---|
| Moist sensation | 4.1 | 4.2 | 4.1 |
| without drying | | | |
| No skin pulling | 4.5 | 4.3 | 4.1 |
| Weight ratio | 1:3 | 1:2 | 2:3 |
| (Trimethylsiloxysili- | | | |
| cate:Polypropyl | | | |
| silsesquinoxane) | | | |

TABLE 6

| Evaluation item | Example 5 | Example 6 | Example 7 |
|---|---|---|---|
| Moist sensation | 4.0 | 3.9 | 3.6 |
| without drying | | | |
| No skin pulling | 3.9 | 3.7 | 3.2 |
| Weight ratio | 3:2 | 2:1 | 3:1 |
| (Trimethylsiloxysili- | | | |
| cate:Polypropyl | | | |
| silsesquinoxane) | | | |

As can be seen from the results in Table 4, Table 5 and Table 6 above, the composition of Comparative Example 2 containing the MQ silicone resin alone showed low satisfaction in terms of drying or pulling, and the composition of Comparative Example 3 containing the polypropyl silsesquioxane resin alone also showed low satisfaction in terms of drying or pulling. However, the composition of Example 1 to 7 containing both the MQ silicone resin and the polypropyl silsesquioxane resin showed high satisfaction in terms of all the evaluation items. Thus, it can be seen that the composition of the present invention, which contains both the MQ silicone resin and the polypropyl silsesquioxane resin, exhibits an excellent moisturizing sensation and the effect of reducing skin pulling.

### TEST EXAMPLE 8 TO 14

### Evaluation of Reduction in Drying and Pulling of the Skin

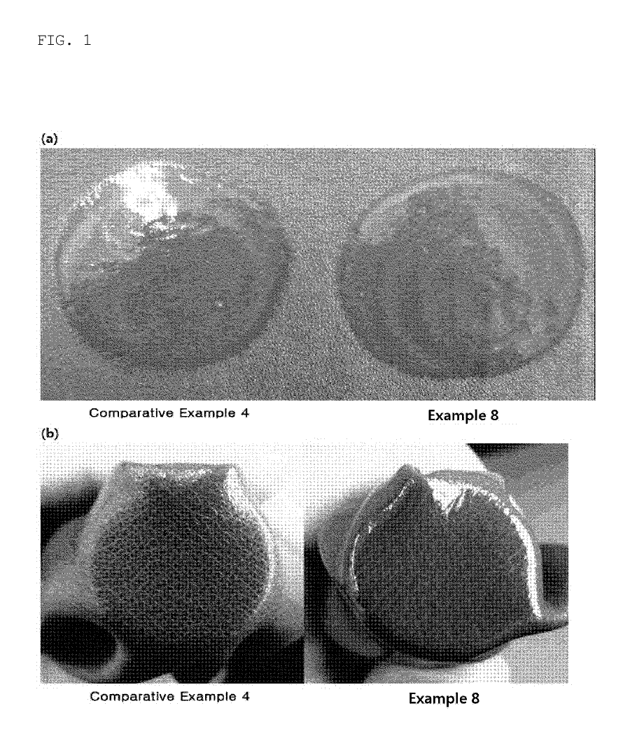

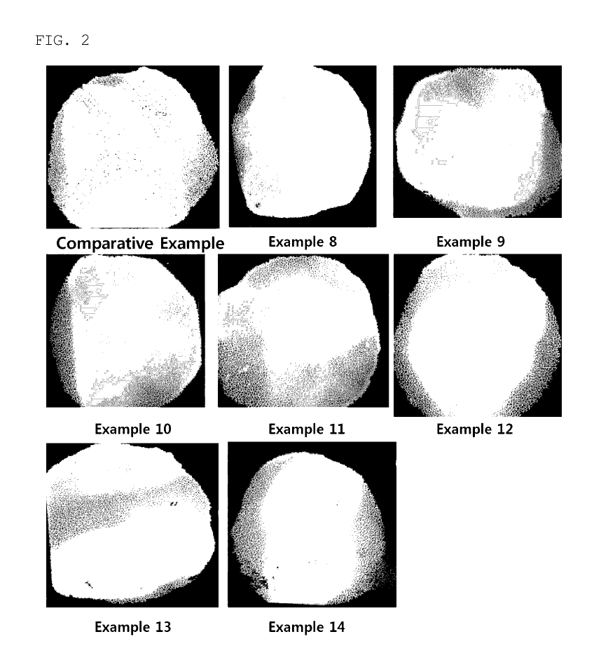

In order to confirm whether drying and pulling of the skin decrease when both the MQ silicone resin and the polypropyl silsesquioxane resin according to the present invention are used, the following test was performed. According to the compositions shown in Table 7 and Table 8 below, powdery components were dispersed in a silicone resin solution. The solution was applied to artificial leather to a thin thickness, and then dried for 15 minutes. The dried synthetic leather was physically stimulated by pulling it in four directions 30 times for each direction, and the surface of the synthetic leather having the solution applied thereto was observed. FIG. 1 and FIG. 2 each shows a set of photographs of the surface of the synthetic leather.

TABLE 7

| | Compar- | | | |
|---|---|---|---|---|
| | ative | | | |
| | Exam- | Exam- | Exam- | Exam- |
| Components (wt %) | ple 4 | ple 8 | ple 9 | ple 10 |
| Trimethylsiloxysilicate | 95 | 47.5 | 23.75 | 31.67 |
| Polypropyl | — | 47.5 | 71.25 | 63.33 |
| silsesquinoxane | | | | |
| Titanium oxide surface | 4.00 | 4.00 | 4.00 | 4.00 |
| treated with alkylsilane | | | | |
| Iron oxide yellow surface | 0.30 | 0.30 | 0.30 | 0.30 |
| treated with alkylsilane | | | | |
| Iron oxide red surface | 0.10 | 0.10 | 0.10 | 0.10 |
| treated with alkylsilane | | | | |
| Iron oxide black surface | 0.07 | 0.07 | 0.07 | 0.07 |
| treated with alkylsilane | | | | |
| Polymethylmethacrylate | 0.53 | 0.53 | 0.53 | 0.53 |
| Weight ratio | — | 1:1 | 1:3 | 1:2 |
| (Trimethylsiloxysili- | | | | |
| cate:Polypropyl | | | | |
| silsesquinoxane) | | | | |

TABLE 8

| | Exam- | Exam- | Exam- | Exam- |
|---|---|---|---|---|
| Components (wt %) | ple 11 | ple 12 | ple 13 | ple 14 |
| Trimethylsiloxysilicate | 38 | 57 | 63.33 | 71.25 |
| Polypropyl | 57 | 38 | 31.67 | 23.75 |
| silsesquinoxane | | | | |
| Titanium oxide surface | 4.00 | 4.00 | 4.00 | 4.00 |
| treated with alkylsilane | | | | |
| Iron oxide yellow surface | 0.30 | 0.30 | 0.30 | 0.30 |
| treated with alkylsilane | | | | |
| Iron oxide red surface | 0.10 | 0.10 | 0.10 | 0.10 |
| treated with alkylsilane | | | | |
| Iron oxide black surface | 0.07 | 0.07 | 0.07 | 0.07 |
| treated with alkylsilane | | | | |
| Polymethylmethacrylate | 0.53 | 0.53 | 0.53 | 0.53 |
| Weight ratio | 2:3 | 3:2 | 2:1 | 3:1 |
| (Trimethylsiloxysili- | | | | |
| cate:Polypropyl | | | | |
| silsesquinoxane) | | | | |

As can be seen from the results in FIG. 1 and FIG. 2, when the surface of the synthetic leather was observed after the synthetic leather was stimulated, it could be seen that, in the case of the composition of Comparative Example 4 containing trimethylsiloxysilicate alone, the surface of the synthetic leather was dried and torn. In other words, it can be expected that the composition of Comparative Example 4 will cause pulling of the skin due to its low flexibility. However, it could be seen that the composition of Example 8 containing trimethylsiloxysilicate and polypropyl silsesquioxane at a weight ratio of 1:1 formed a moist flexible layer without tearing or splitting of the surface of the synthetic leather.

Also, it could be seen that the composition of Example 9 to 14 containing trimethylsiloxysilicate and polypropyl silsesquioxane at a weight ratio of 1˜3:1˜3, preferably 1:3, 1:2, 2:3, 3:2, 2:1 and 3:1, and most preferably 1:3, 1:2 and 2:3 formed a moist flexible layer without significant tearing or splitting of the surface of the synthetic leather.

Although the extent of tearing of the surface of the synthetic leather were increased by increasing the amount of Trimethylsiloxysilicate in the composition of Example 12 to 14, the tearing effects were far less than Comparative Examples 4 which showed a higher tendency of cracking as shown in FIG. 1 and FIG. 2. Hence, we concluded that a moist flexible layer was formed on the surface of the synthetic leather with the composition of Example 8 to 14.

### INDUSTRIAL APPLICABILITY

The makeup cosmetic composition according to the present invention contains, in addition to MQ silicone resin, a siloxane resin exhibiting an excellent moisturizing sensation without pulling of the skin, and thus can maintain the skin in a moist state without drying and pulling of the skin.

1. A makeup cosmetic composition, which contains MQ silicone resin and propyl silsesquioxane resin,
wherein the MQ silicone resin comprises a siloxy unit, selected from an M unit represented by (R₃¹SiO_{1/2})ₐ and a Q unit represented by (SiO_{4/2})_{b}, in an amount of 80 mole % or more based on the total moles of the MQ silicone resin, in which the R¹ groups are independently an alkyl group having 1 to 8 carbon atoms or an aryl group, and a and b are integers greater than 0;
wherein the propyl silsesquioxane resin comprises a T unit, represented by R²SiO_{3/2}, in an amount of 80 mole % based on the total moles of the propyl silsesquioxane resin, in which the R² groups are independently an alkyl group having 1 to 8 carbon atoms or an aryl group, and 40 mole % or more of R² is propyl;
wherein the MQ silicone resin and the propyl silsesquioxane resin are contained at a weight ratio of 1˜3:1˜3; and
wherein the composition further contains as an additive:
polymethylmethacrylate; and
titanium oxide surface treated with alkylsilane or a mixture of titanium oxide surface treated with alkylsilane and iron oxide surface treated with alkylsilane;
wherein the iron oxide is yellow, red or black.

2. The makeup cosmetic composition of claim 1, which is used for reducing skin pulling and/or increasing skin moisturization.

3. The makeup cosmetic composition of claim 1, wherein 80 mole % or more of the MQ silicone resins consists of the M and Q units.

4. The makeup cosmetic composition of claim 1, wherein the propyl silsesquioxane resin further contains one or more selected from the group consisting of M, D and Q units.

5. The makeup cosmetic composition of claim 1, wherein the MQ silicone resin is contained in an amount of 0.01-30 wt % based on the total weight of the composition.

6. The makeup cosmetic composition of claim 1, wherein the propyl silsesquioxane resin is contained in an amount of 0.01-30 wt % based on the total weight of the composition.

7. The makeup cosmetic composition of claim 1, wherein the MQ silicone resin and the propyl silsesquioxane resin are contained at a weight ratio of 1:3, 1:2, or 2:3.

8. The makeup cosmetic composition of claim 1, wherein the composition is of a type selected from the group consisting of a water-in-oil type, an oil-in-water type and an oil dispersion type.

9. A method of increasing skin moisturization or reducing skin pulling, the method comprising applying a cosmetic composition to skin of a subject in need of skin moisturization or reduction of skin pulling, the composition containing MQ silicone resin and propyl silsesquioxane resin as an effective ingredient,
wherein the MQ silicone resin comprises a siloxy unit, selected from an M unit represented by (R₃¹SiO_{1/2})ₐ and a Q unit represented by (SiO_{4/2})_{b}, in an amount of 80 mole % or more based on the total moles of the MQ silicone resin, in which the R¹ groups are independently an alkyl group having 1 to 8 carbon atoms or an aryl group, and a and b are integers greater than 0;
wherein the propyl silsesquioxane resin comprises a T unit, represented by R²SiO_{3/2}, in an amount of 80 mole % based on the total moles of the propyl silsesquioxane resin, in which the R² groups are independently an alkyl group having 1 to 8 carbon atoms or an aryl group, and 40 mole % or more of R² is propyl;
wherein the MQ silicone resin and the propyl silsesquioxane resin are contained at a weight ratio of 1˜3:1˜3; and
wherein the composition further contains as an additive:
polymethylmethacrylate; and
titanium oxide surface treated with alkylsilane or a mixture of titanium oxide surface treated with alkylsilane and iron oxide surface treated with alkylsilane;
wherein the iron oxide is yellow, red or black.

10. The method of claim 9, wherein the titanium oxide treated with alkylsilane is contained in an amount of 4 to 8 wt % based on the total weight of the composition, the iron oxide treated with alkylsilane is contained in an amount of above 0.05 to under 1 wt % based on the total weight of the composition; and the polymethylmethacrylate is contained in an amount of above 0.5 to under 2.0 wt % based on the total weight of the composition.

11. The method of claim 9, wherein 80 mole % or more of the MQ silicone resins consists of the M and Q units.

12. The method of claim 9, wherein the propyl silsesquioxane resin further contains one or more selected from the group consisting of M, D and Q units.

13. The method of claim 9, wherein the MQ silicone resin is contained in an amount of 0.01-30 wt % based on the total weight of the composition.

14. The method of claim 9, wherein the propyl silsesquioxane resin is contained in an amount of 0.01-30 wt % based on the total weight of the composition.

15. The method of claim 9, wherein the composition is of a type selected from the group consisting of a water-in-oil type, an oil-in-water type and an oil dispersion type.
